(19) United States
(12) Patent Application Publication
(10) Pub. No.: US 2007/0078523 A1
(43) Pub. Date: Apr. 5, 2007

(54) Lower limb prosthesis

(76) Inventors: Deepesh K. Kholwadwala, Albuquerque, NM (US); Lubov V. Slepova, Snezhinsk (RU); Jason W. Wheeler, Albuquerque, NM (US); Kamyar Rahimian, Albuquerque, NM (US); Brandon R. Rohrer, Albuquerque, NM (US); Gabriel A. Johnston, Trophy Club, TX (US); Jennifer L. Coleman, Belen, NM (US); Mark R. Vaughn, Albuquerque, NM (US); Vladimir I. Pervushin, Snezhinsk (RU); Anatoli S. Ivanov, Snezhinsk (RU)

Correspondence Address: SANDIA CORPORATION, P O BOX 5800, MS-0161, ALBUQUERQUE, NM, 87185-0161, US

(21) Appl. No.: 11/527,143

(22) Filed: Sep. 26, 2006

Related U.S. Application Data
(60) Provisional application No. 60/723,219, filed on Oct. 3, 2005.

(51) Int. Cl. A61F 2/80; A61F 2/62; A61F 2/66

(52) U.S. Cl. 623/36; 623/33; 623/38; 623/55

(57) ABSTRACT

A lower limb prosthesis comprising a foot, a pylon, a socket, a connector between the socket and the pylon, an adapter between the foot and the pylon, and a liner in the socket, the liner being substantially rigid at room temperatures and moldable at a temperature between approximately 50 degrees C. and approximately 100 degrees C. Also a support for a residual lower limb, the support being sized to receive the residual lower limb and comprising a socket and a liner in the socket, the liner being substantially rigid at room temperatures and moldable at a temperature between approximately 50 degrees C. and approximately 100 degrees C.

### CROSS-REFERENCE TO RELATED APPLICATIONS

This application claims priority to and the benefit of the filing of U.S. Provisional Patent Application Ser. No. 60/723,219, entitled “Economical Third World Lower Limb Prosthesis”, filed on Oct. 3, 2005, and the specification and any claims thereof are incorporated herein by reference.

### STATEMENT REGARDING FEDERALLY SPONSORED RESEARCH OR DEVELOPMENT

The Government has rights to this invention pursuant to Contract No. DE-AC04-94AL85000 awarded by the U.S. Department of Energy.

### BACKGROUND OF THE INVENTION

The present invention relates to lower limb prostheses. The present invention additionally relates to lower limb prostheses that are adjustable to accommodate the changing needs of an individual user. The present invention additionally relates to standardized lower limb prostheses that can be adjusted to accommodate a wide variety of user shapes and sizes.

Currently available prostheses are built (e.g., custom cast and fitted) for one person only and are not adjustable once they have been fabricated. Should the residual limb change shape or a juvenile amputee grow, the amputee must obtain a completely new prosthesis.

The present invention provides a prosthetic device that is fully adjustable. For example, if a juvenile amputee should grow, the foot and pylon of embodiments of the invention can be lengthened to adjust for these changes. Additionally, residual limbs change shape by the hour. These changes are generally not accounted for in currently available prostheses and can cause a great deal of pain over a relatively short period of time. In embodiments of the present invention, these changes can be adjusted for by simply heating up an inner liner of the socket and impressing a new shape on the socket. Once the limb settles down and stops changing shape, the socket can be left at the most comfortable configuration for a longer period of time.

Furthermore, in the Western world, prosthetic devices can cost up to $50,000 to produce. Often, these devices need to be replaced every year due to changes in the shape of the residual limb. These “western” limbs are far too expensive for use in less developed countries.

Embodiments of the invention allow the amputee to adjust the shape of the socket whenever there is a change in the shape of his/her residual limb, allowing for a closer and more comfortable fit of the prosthetic over a longer period of time. The invention is inexpensive to maintain as well as better able to handle the rigors of use in underdeveloped countries or areas.

### BRIEF SUMMARY OF THE INVENTION

The present invention is of a lower limb prosthesis comprising: a foot; a pylon; a socket; a connector between the socket and the pylon; an adapter between the foot and the pylon; and a liner in the socket, the liner being substantially rigid at room temperatures (from approximately −24 degrees C. to approximately 40 degrees C.) and moldable at a temperature between approximately 50 degrees C. and approximately 100 degrees C. In the preferred embodiment, the socket comprises a plurality of upward extending wings cantilevered at one end of the socket, preferably wherein one of the wings is substantially wider than all other of the wings. The liner is moldable at a temperature between approximately 50 degrees C. and approximately 65 degrees C. The liner comprises an acetate or acrylic acid copolymer, preferably a copolymer selected from ethylene/acrylic acid, ethylene/methacrylic acid, and ethylene/vinyl acetate, preferably ethylene/vinyl acetate, more preferably wherein the liner comprises between 10% and 50% vinyl acetate by weight, and most preferably wherein the liner comprises approximately 40% vinyl acetate by weight. The liner has a melting temperature of approximately 150 degrees C. or higher. The foot comprises a releasable rocker mechanism, the rocker mechanism operatively configured to allow a wearer to squat, and the connector is adjustable for proximal, distal, and lateral alignment.

The present invention is further of a support for a residual lower limb, the support being sized to receive the residual lower limb and comprising a socket and a liner in the socket, the liner being substantially rigid at room temperatures (from approximately −24 degrees C. to approximately 40 degrees C.) and moldable at a temperature between approximately 50 degrees C. and approximately 100 degrees C. In the preferred embodiment, the socket comprises a plurality of upward extending wings cantilevered at one end of the socket, most preferably wherein one of the wings is substantially wider than all other of the wings. The liner is moldable at a temperature between approximately 50 degrees C. and approximately 65 degrees C., comprises materials as described in the preceding paragraph, and has a melting temperature of approximately 150 degrees C. or higher. The support additionally comprises a foot comprising a releasable rocker mechanism, the rocker mechanism operatively configured to allow a wearer to squat and a pylon and a connector between the socket and the pylon, the connector being adjustable for proximal, distal, and lateral alignment.

Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 is a perspective view of the preferred embodiment of the invention with liner and suspension sleeve removed;

FIG. 2 is a perspective view of an embodiment of the socket shell of the invention with the liner removed;

FIG. 3 is a perspective cut-away view of another embodiment of the socket shell;

FIG. 4 is a perspective view of the alignment plate for proximal and distal adjustments;

FIG. 5 is a perspective view of the adapter for connection between the pylon and socket;

FIG. 6 is a perspective view of the assembly of the items of FIGS. 4-5 including a washer;

FIGS. 7(a) and (b) are perspective views of the two primary components of FIG. 5;

FIGS. 8(a)-(c) are perspective views of an embodiment of the pylon of the invention, including knee joint and ankle joint;

FIG. 9 is a perspective view of an embodiment of the foot of the invention;

FIG. 10 is a perspective view of another embodiment of the foot of the invention;

FIG. 11 is a perspective view of an embodiment of the suprapatellar strap;

FIG. 12 is side view of an embodiment of the suspension cuff and tension system;

FIG. 13 is a perspective view of a liner according to the invention; and

FIG. 14 is a perspective view of the liner situated in the socket shell of the invention.

### DETAILED DESCRIPTION OF THE INVENTION

The present invention comprises a low-cost, one-size-fits-all, lower limb prosthesis that includes an adjustable socket with an adjustable liner for receiving a stump, a height adjustable pylon and a length adjustable foot. The invention allows a manufacturer to mass-produce these devices for primarily overseas markets. The preferred design of the prosthetic allows untrained users to fit and re-fit the device “in the field” without the need for specialized prosthetic-fitting technicians, and without the need for custom molding/casting of components to a specific user at an instant in time. This achieves one-size-fits-all and allows for “stocking” of multiple units for immediate deployment to the field. This additionally provides for re-fitting the prosthetic as the size, shape or needs of the user changes over time.

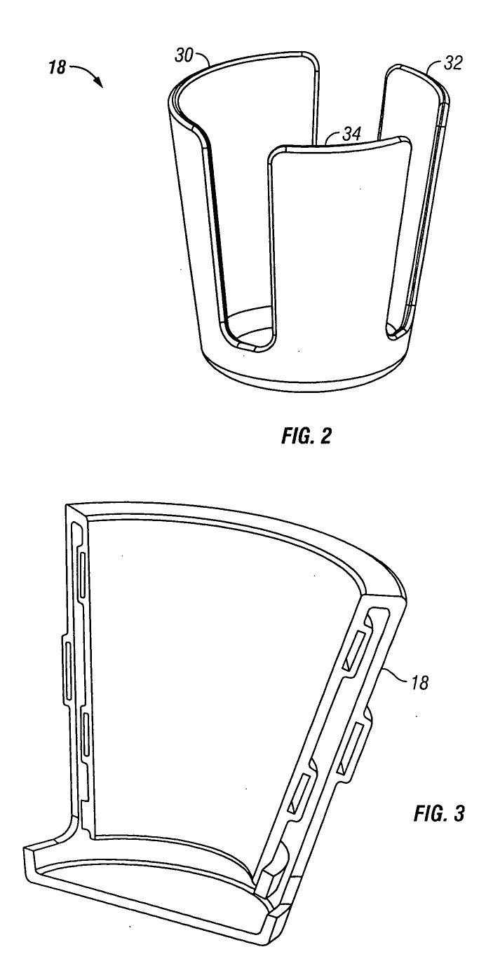

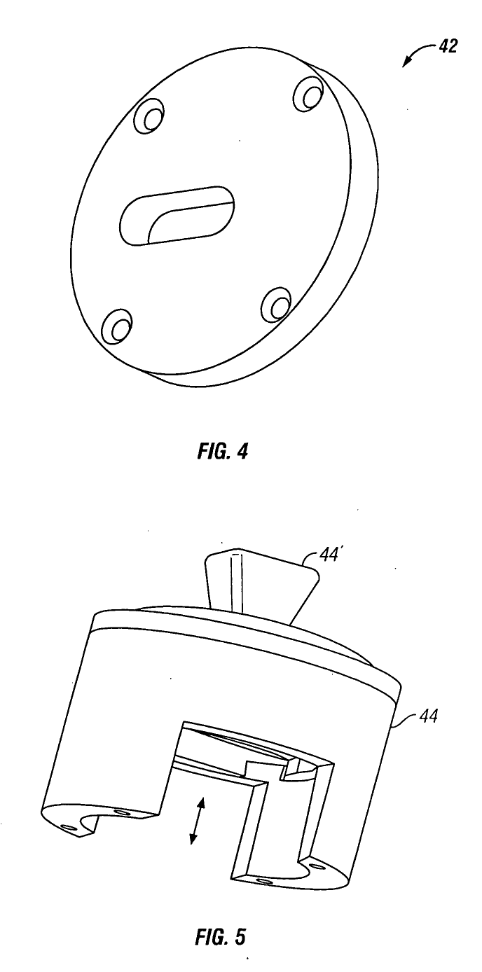

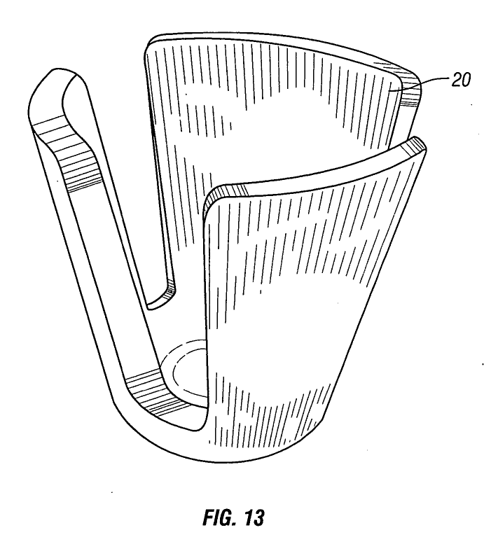

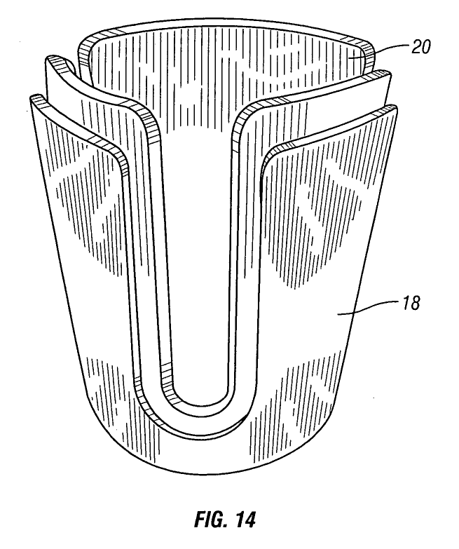

The components of the prosthetic can be fastened together with adjustable couplings that provide for adjustments to be made, for example in the alignment of the components to match the users shape and stance. The socket is presented in two different preferred configurations. A simple cup shape (see FIG. 2) with wings provides one configuration, and a change to the base cup shape where the wings are cantilevered at one end of the socket for improved fit provides a second configuration (see FIG. 3). The socket can contain a liner (see FIGS. 13 and 14) of a moldable material that can be shaped and re-shaped by heating at temperatures low enough to allow using heated water, but below temperatures that would injure a patient. Thus, above normal use temperatures, the liner material is used to obtain a precise fit between the stump and socket.

Two different preferred foot configurations are provided, given that both incorporate an internal spring element to provide energy return. The second design provides a steel stop that can be released to accommodate squatting.

As noted above, current prosthetic limbs are manufactured to fit each individual person. The Universal Limb of the invention provides a low cost solution to this problem. Its simplicity allows less skilled people to assemble, adjust, and maintain it without special tools. The advantages of this limb over those already produced include its off-the-shelf simplicity, adjustable liner and pylon, tough foot, flexible ankle, and maintenance-free parts. The Universal Limb is designed to be adjustable for different people. This is achieved preferably by using a socket liner that can be heated and molded to fit each person. When the residual limb changes shape, the socket can be remolded, and a close fit maintained without the need for a trained professional. Not only does such reshaping provide a better fit for multiple limb shapes, but it also provides extra stability due to the complete contact between socket, liner, and limb. This improves the user's control of the prosthetic device.

A preferred polymer has been identified for this application. It is non-deformable at room temperature (preferably approximately −24 degrees C. to approximately 40 degrees C.), but moldable at a temperature not far above body temperature. The liner is reshaped by heating it in hot water, and then placing the socket with the liner on the residual limb for shaping. This material is resistant to water, oil, and salt, a necessity due to the proximity of the skin. It is also biocompatible with human tissue. The prosthesis will typically be used outdoors, and is therefore abrasion resistant and water resistant.

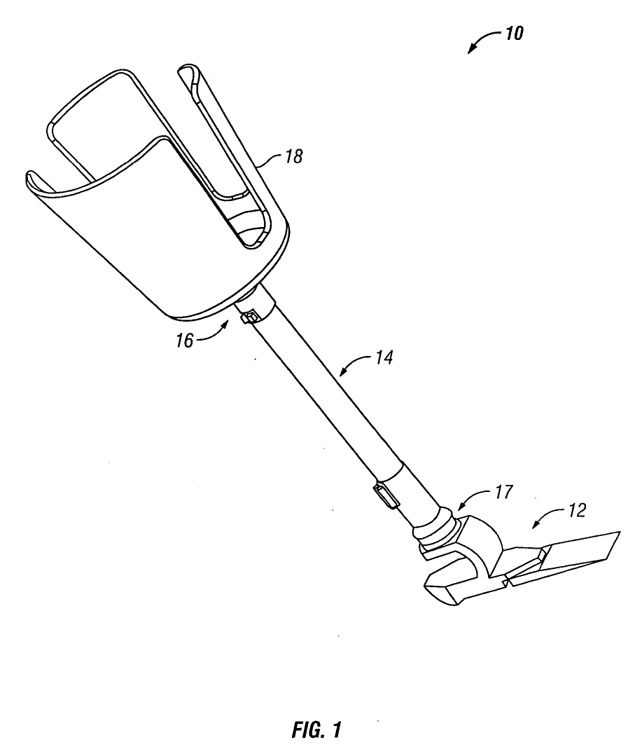

As shown in FIGS. 1 and 12-14, there are six different parts preferred that comprise the Universal Leg 10: foot 12, pylon 14 (shin), connector 17, adapter 16, socket 18 (alternative embodiment in FIG. 3), in which will be located liner 20 (see FIGS. 13 and 14), and suspension 22 (see FIG. 12). When assembled, these parts should enable the amputee to come quite close to mimicking natural human motion. They should also be adjustable so that changes can be made as the residual limb changes. In these adjustments, the need for tools is kept to a minimum. All parts and tools are inexpensive, yet durable enough to last many years without replacement, even in a harsh environment. The preferred components are described below.

The basic structure of the human foot involves a series of bones and muscles which are connected by tendons. This complex system allows for smooth gait and minimal energy loss during ambulation. If designed correctly, the prosthetic foot should simulate the human foot as closely as possible. The foot should rebound energy back into the stride while helping to bring the foot back to proper initial alignment during the recovery phase of walking. This mirrors the function of muscles and tendons. Integrated into the foot is an ankle which will allow for squatting and sitting cross legged.

The pylon mimics the human shin bone. In a fully adjustable prosthesis, it should adjust for various heights so that as a young amputee grows, the prosthesis can grow with him. The pylon should flex a little with the stride of the amputee, but excess bending can cause pressure sores on the residual limb due to torsional forces, and must be avoided. The pylon must also be adjustable in posterior/anterior/lateral directions to obtain correct alignment. This is accomplished by the female adapter of the invention.

The connector is the interface between the pylon and the socket. This connector should be universal and durable. Should the prosthetic need to be replaced, the pylon and foot of the leg can be kept, and a new socket can be added. This will occur if the initial socket was not built properly or is broken and needs to be replaced. Because of the connectors and adapters, the parts that last longer then others will be kept, and the overall cost of replacement will be kept down.

The outer socket (shell) should be tough, abrasion resistant, water resistant, UV resistant, and somewhat flexible. It will have a larger diameter than the largest residual limb of the amputee population so that a liner can be used to adjust the size for smaller or larger limbs.

The liner (inner socket) must also be abrasion resistant, water resistant, and adjustable. Ideally, the substance used for the liner should be soft when warmed in hot water and somewhat hard when cooled. In this way, the amputee can reshape the socket to fit the residual limb exactly. Since the residual limb changes over time, the liner should be resilient enough to be changed numerous times before wearing out.

The suspension system should comfortably, yet securely, hold the prosthesis to the residual limb. This should happen without restricting blood flow, inhibiting smooth walking or pinching the amputee. This system should be redundant so that if one method of suspension fails, another can be used so the amputee is never without the limb.

During the course of analyzing liner materials, a model of the actual limb was being designed. Numerous designs were made and analyzed for their advantages and disadvantages. The first design was a solid shell with an adjustable liner inside. A solid shell does not allow for adjustment should the residual limb be too large, so a few conical wing designs were created.

There are many issues that need to be considered when designing a socket. An important issue is where weight is distributed on the limb. As explained above, certain areas of a residual limb are more susceptible to injury due to consistent pressure in that area. Weight on the gastrocnemius and soleus muscles generally does not result in ulcers on the limb, but weight on the shin bone and distal end of the limb can be quite painful as well as being the cause of lesion formation. Due to these issues, the three wing design is preferred over a solid-walled shell. In this design, the majority of the weight rests on larger muscle groups, leaving the more sensitive areas free from pressure. The design with three wings of equal width is less preferred than a design with the rear wing being wider than the front wings. This allows the weight to be distributed where padding will be the most beneficial. The cone design is preferred because the shape of the ideal limb after amputation is conical. This design also prevents pinching between the wings when the residual limb is smaller than the prosthetic socket.

The preferred design comprises a one-piece shell made from ¼ inch thick sheets of a polypropylene/polyethylene copolymer. This material has qualities that make it ideal for this shell. First, it is stiff. It provides the necessary backbone for the soft liner. It is compatible with the preferred Ethylene/Vinyl Acetate copolymer that is used for the liner. Epoxies can be used to glue the liner to this shell and are inexpensive. In order to shape this material, it is heated in an oven until soft and wrapped around a mold. A vacuum is applied and creates an exact model of the outside of the mold. This material can be fully melted and formed by injection molding.

FIG. 2 illustrates the preferred shell absent a liner. Note the shape of the wings 30,32,34, the cutouts, and how tall the wings are compared to the solid bottom part of the sides. The wings were left as wide as possible in order to provide the most surface area for the liner to be in contact with the skin of the residual limb. The cutouts are preferably straight lines, and are relatively easy to mold in mass production. The wings are tall (cutouts deep) in order to allow more flexibility in the wings for adjustment to the diameter of the socket. The residual limb is used to add structure to the limb overall. There are preferably lacing holes (not shown) drilled up each side of the wings which are used in the event of the outer compression cuff wearing out. In this case, the user can obtain fasteners and tie the limb on until a replacement cuff can be obtained.

The socket shell and overall socket design progressed from a solid outer shell through wrap around designs to winged options. Overall, it was decided that the winged options allowed the best ventilation for skin health as well as better adjustability overall. There were four different variations on the winged socket shell. The first idea was a four-winged shell. The front was cut down to fit below the patella while the sides remained high in order to gain stability from the knee (condyles) and thigh. The back was cut down to fit behind the fold of the knee.

FIG. 3 illustrates a cantilevered winged shell alternate that provides further adjustability at the lower end of the residual limb. Each wing is cantilevered at one end of the socket. In the simpler version in FIG. 2, radial gaps between the residuum and the shell near the bottom of the shell can be taken up by the liner only, thus potentially allowing motion of the tip of the residual limb. This can cause abrasion leading to pressure sores. The shell in FIG. 3 restrains the tip of the stump by allowing the inner flaps (i.e., cantilevered wings) to be separately adjusted with a strap while the middle and top of the stump is restrained by two other straps. This feature allows further adjustability of the socket and thus allows the limb to be used by a wider variety of amputees.

The liner of the invention has unique requirements. The liner is the interface between the shell and the residual limb. For the best control, the liner needs to be in complete contact with both the shell and the residual limb. It should not put pressure on small or bony areas of the leg, but instead, pressure should be evenly distributed over the entire surface of the limb. For simplicity, the Universal Limb should be user friendly, allowing adjustments to be made by the user instead of by a prosthetist. In order to make this possible, the liner geometry needs to be changeable in a simple manner. The liner does not need to provide all of the support for this prosthesis. The shell has been designed to fulfill that purpose.

The purpose of the preferred polymer liner is to act as a pressure relief mechanism. It makes the socket fully adjustable so the socket will fit various sizes and shapes of residual limbs. Excess polymer can be cut away, molded like clay, and added in a different area where it is needed in order to reshape the socket. Not only does this reshaping provide a better fit for multiple limb shapes, but also provides extra stability due to the close contact between socket and limb. This improves control of the prosthetic device. The liner is designed to be the unifying structure between the hard straight outer shell (i.e., socket) of the prosthesis and the soft round skin of the residual limb. It allows for uniform pressure to be applied over the entire residual limb instead of on a few pressure bearing points.

A particular polymer was formulated to fill this need. The material must be indeformable (i.e., substantially rigid) at room temperatures (from approximately −24 degrees C. to approximately 40 degrees C.) but moldable at a temperature not far above body temperature. A simple way to get even heating across a large surface area without scorching another surface is by using hot water. Since water is generally available as are methods for heating water, reshaping is done by heating the piece in hot water and placing it on the limb to shape it. The residual limb of a new amputee is quite tender, even with an insulating sock, so this reshaping temperature cannot be too high. In order to get this low reshaping temperature, the glass transition temperature of the polymer must be low as well, certainly below 100 degrees C., and most preferably between 50 and 65° C.

The prosthesis will be used outdoors, and so needs to be abrasion resistant and water resistant. Resistance to sweat and oil is also a requirement due to its proximity to the skin. A substance that has been approved as biocompatible by the Food and Drug Administration or equivalent bodies outside the United States is preferred.

At human body temperature the polymer should be somewhat hard. Slightly above body temperature, but below burning temperature for the skin, this polymer should soften, allowing it to be reshaped to the features of the limb without causing excess pain to the amputee. The melting point of this material should be above the boiling point of water, a temperature that can not be easily reached by the amputee. This melting temperature allows the copolymer to be molded while it is in a liquid state. Physical decay of this copolymer should not occur until well above the boiling point of water, preferably about 300°F.

Not only are there thermal requirements for the socket, but there are also requirements for the shape of the liner. These same requirements were mirrored in the socket. Each residual limb has its own unique shape, but there are similarities between each limb that need to be relied on in order to make a leg that is useful to the greatest number of people. Some of these are: conical shape; rounded tip; liner made of a polymer that can be reshaped easily; different sizes of cone for different limb sizes; and a polymer that can be reshaped using hands and stump, no tools except a pot, a fire, and some water. Since there will typically be no prosthetist present to assist the amputee with this limb after the initial fitting, liner maintenance must be simple. Maintenance requirements of the liner include: easily cleanable; flexes with the residual limb; nothing technical about adjusting the socket, no measurement, just boil and shape; able to be molded from original shape to either larger or smaller stumps; and able to handle sun exposure, stretching stresses, dirt, washing, and accidental cutting.

A range of materials useful for the present invention are Ethylene/Acrylic Acid and Ethylene/Vinyl Acetate copolymers, preferably already polymerized, which only need to be melted down and cast into the desired shape. They are preferably also stabilized, which gives them a long shelf life. The ethylene compounds of Table I use a polyethylene mixed with an acrylic acid compound to make a copolymer. The polyethylene is a hard plastic, and the acrylates dissolve it, turning it into a softer compound that has the softening ability of the acrylate and the hardening ability of the polyethylene. It retains its shape when it is cold, but when it is warmed up, it can be reshaped. Certain heat-formable mouth guards are made from Ethylene/Vinyl Acetate, a copolymer made from Ethylene and the Vinyl Acetate monomer.

TABLE 1
Ethylene Compounds

| # | Compound Name | Make-up | Concentration | Melt Index |
|---|---|---|---|---|
| E1 | E/AA | Copolymer | 5% AA | |
| E2 | E/AA | Copolymer | 9% AA | |
| E3 | E/MA | Ionomer Zn | | MI = 1.6 |
| E4 | E/MA | Ionomer Zn | | MI = 5.5 |
| E5 | E/MA | Ionomer Zn | | MI = 14 |
| E6 | E/MA | Ionomer Na | | MI = 0.9 |
| E7 | E/MA | Ionomer Na | | MI = 2.8 |
| E8 | E/MA | Ionomer Na | | MI = 10 |
| E9 | E/MA | Copolymer | 4% MA | |
| E10 | E/MA | Copolymer | 9% MA | |
| E11 | E/MA | Copolymer | 12% MA | |
| E12 | E/VA | Copolymer | 14% VA | |
| E13 | E/VA | Copolymer | 40% VA | |
| E14 | E/VA | Copolymer | 25% VA | |
| E15 | E/VA | Copolymer | 18% VA | |

Table 1 shows a list of all the ethylene compounds that were tested in a set of experiments. In order to understand the table, each of the acronyms must be explained. In the first column, the E says that the sample is an ethylene compound. The following numbers tell one which of the copolymers that sample is made of. In the second column, there are many different compounds listed. AA stands for Acrylic Acid. MA stands for methacrylic acid. VA stands for Vinyl Acetate. The third column explains the makeup of the copolymer, whether a copolymer or an ionomer. Each of the ionomers is followed by which ion is used. The last columns give either the viscosity of the polymer when melting (known as the Melt Index) or the percentage of acrylate in the mixture.

The 40% Ethylene/Vinyl Acetate (“EVA”) is preferred due to its rubbery consistency when cool and its ease in forming when hot. This mix of EVA does not require lots of heat to shape and cools quickly, yet when cool, does not become so hard as to be uncomfortable. It also meets the other requirements of the preferred liner.

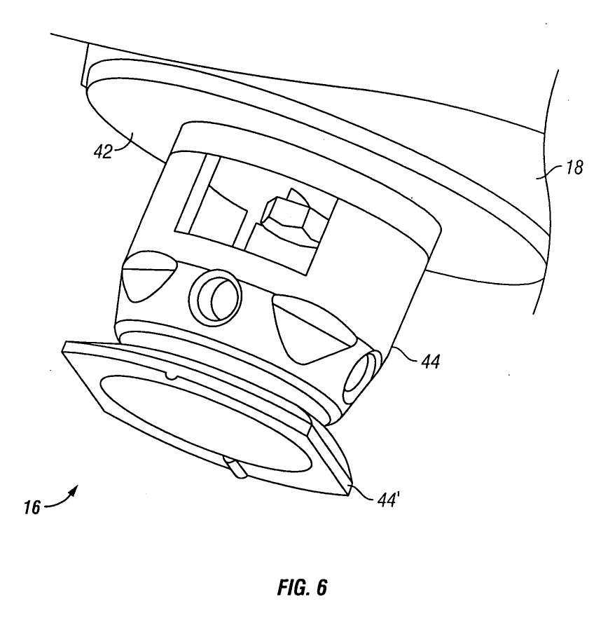

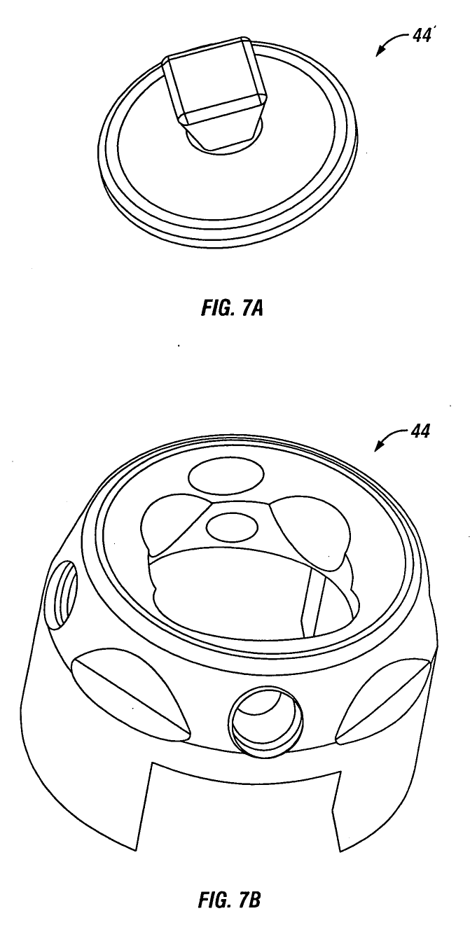

In order to connect the pylon 14 to the socket 18, a connector 16 is necessary. This connector is more aptly described as an adapter because it not only connects the pylon to the socket, but it also allows for proximal, distal and lateral alignment changes to be made. For ease of interface between components of this limb and other limbs on the market, standard connectors are preferred. Additional pieces were added to these adapters in order to make the adjustable alignment option feasible. These are shown in FIGS. 4-6, including alignment plate 42 (illustrating a slotted alignment feature) and female adapter 44 with male pyramid adapter 44′ for connection between the pylon and socket.

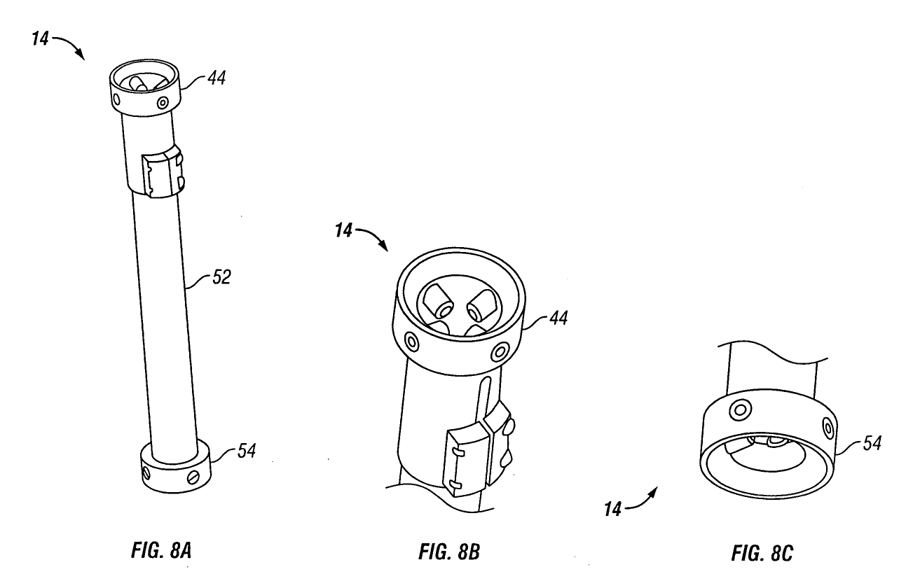

There are other adapters needed in a prosthetic besides the one that connects the socket and the pylon. The most important of these adapters is adapter 17 between the ankle and the pylon 14. The same adapter component 44′ is used on the base of the socket 18 as is used on the top of the ankle. FIGS. 7(a) and 8(c) show the preferred version of this adapter.

Again, these adapters are designed to make alignment of the prosthesis simple. This way, all prosthetic components can be traded for what is on this limb and vice-versa. Each of the parts is preferably made from 7075-T6 or 6061-T6 Aluminum Alloy except the male pyramid adapter which is preferably made from 6AL-4V Titanium.

The pylon 14 and foot 12 of the prosthetic constitute the remainder of the limb. Due to the increasing height of growing children, the length of the pylon (shin) should be adjustable. It should be sturdy, yet light, capable of handling the rotational and compressional motion of human gait. The ends of the pylon should have connectors that easily attach to the pyramid connectors of the socket and foot.

The pylon 14, the replacement for the shin, has three main components (FIGS. 8(a)-(c)). A cylindrical tube 52 preferably of titantium is connected to the foot with a titanium adapter 44. This can be adjusted a few inches to add length to the cylinder. The other end of the cylinder is connected to the socket with a titanium sleeve and set screws 54.

The titanium tube is preferably 1.5″ in diameter and can be cut to any length for the purposes of the amputee (it preferably starts out at about 2.5′ long). This tube can be replaced with other materials should the amputee grow and require a longer pylon. At each end of the tube is an adapter. Both adapters are generally cylindrical in nature although there are features built on to the sides that allow for clamping functions to take place.

The adapter on the end near the socket is the shorter of the two, and has one side which fits into the pylon by compression fit. This adapter is not adjustable on the compression fit side. The other side of the adapter is larger in diameter and preferably has four set screws in the walls which serve to connect the pylon to the male adapter as shown in FIG. 7a.

The second adapter (near the foot) is adjustable for length, allowing a bit of growing room for the amputee before the pylon tube needs to be replaced. This adapter clamps on the outside of the pylon tube using a compression fit generated by the two screws and four nuts machined into the side of the adapter (See FIG. 8b). The length of the pylon can be adjusted by loosening the screws and sliding the adaptor over the tube. A tool to adjust this adapter comes with the limb. The other side of this adapter is the same as the second side of the previous adapter, allowing for attachment to the male adapter shown in FIG. 7a.

One of the most important components of the prosthesis is the foot 12. The characteristics of the foot can make a prosthesis simple to use or can introduce forces that make the prosthesis impossible to use. The main components of a prosthetic foot that need to be integrated into a design are energy return and flexibility for squatting. The foot needs to feed energy back into the walking system. In most simple prosthetic legs, the limb saps energy from the user at every step. If this foot can return energy with an internal spring, the amputee would be able to use the leg for a longer period of time before reaching the point of exhaustion.

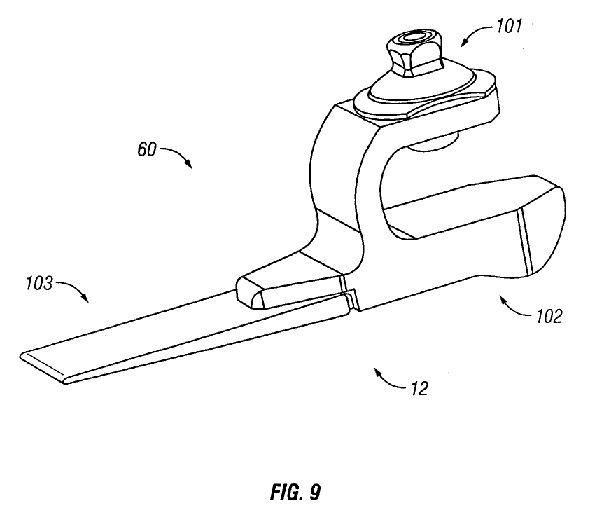

Two foot designs are presented. The first 60 is a simple, lightweight foot with a compliant member to provide dynamic ankle flexion (see FIG. 9). The structure of the foot is preferably made of lightweight polyamide. The adapter and the bolt are preferably steel. The length of the foot can be adjusted with simple tools to fit the needs of the patient. This foot would provide moderate functionality at a low cost and weight.

The C design that forms the heel and ankle of this foot design are useful for two purposes. First, as the heel strikes the ground, the bottom of the C acts as a shock absorber. This shock absorber absorbs the energy of the amputee's forward motion. As the amputee rocks forward onto the forefoot, this energy moves around the curve of the C, and is released into the pylon of the prosthesis, helping to propel the amputee forward. As the amputee puts weight on the forefoot, energy is also stored here and released upon the toe off portion of the stance cycle.

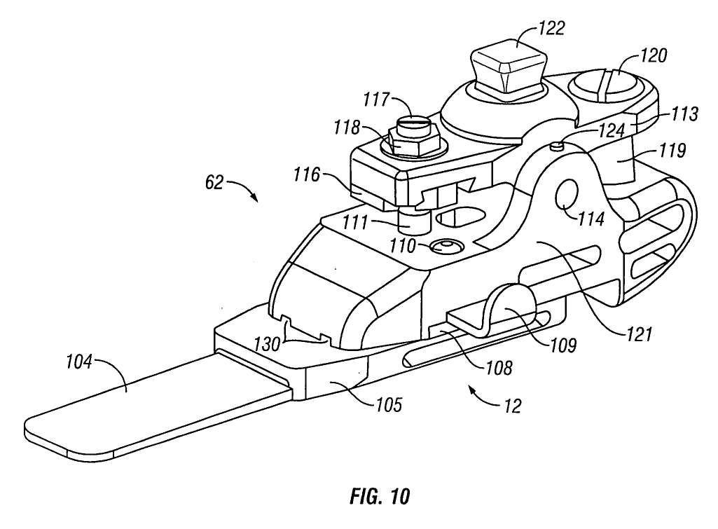

The second foot design 62 offers increased functionality with additional cost and weight (see FIG. 10). This foot has a hinged ankle with a rubber stop mounted on the heel which provides dynamic cushioning. The foot is preferably made of polyamide and the ankle rocker mechanism is preferably aluminum. There is a steel stop in front of the ankle which can be released. This allows additional dorsiflexion, accommodating squatting. The length of the foot is adjustable, just like the first foot 60.

The heel of this foot absorbs energy in the same manner as the first foot did, but releases that energy through the rubber bumper to the pylon, propelling the user forward. After mid-stance, the energy of the stride is stored in the forefoot, and released in the same manner as the first foot. The main advantage to this foot is the ability to release the ankle for unhindered squatting. This could be an advantage in some farming based cultures.

The materials used to make these feet are unreactive, resistant to UV radiation, tough, and flexible. They could be used by themselves as the foot of a prosthesis. With a simple rubber cosmesis, these feet could be made to look natural, easily fitting inside a shoe. The addition of the rubber foot cosmesis would make the prosthetic foot last longer.

The foot shown in FIG. 9 preferably comprises three pieces. Part 101 is the ankle component of the foot. This is composed of a commercial male adapter shown in FIG. 7A. This mounts on the top of the “heel” of the foot which is the shock absorbing part of the foot. This “heel” (part 102) is composed of a “h” shaped piece of polyamide plastic laid on its back. The male adapter mounts on the shorter of the vertical parts of the “h”. The curly part of the “h” acts as the shock absorber, compressing and tightening the curve upon heel strike and opening back up again, returning some energy back to the amputee as the limb progresses towards toe-off in the stance cycle. The heel part of the “h” has a rounded shape to make the heel strike and ensuing roll forward a smooth motion. The vertical of the “h” shaped part has a groove cut in it that accepts a “tongue” on the “toe” part of the foot. Part 103 is the toe piece which is a “tongue” half and a “toe” half. The Tongue half has a slot cut into it that is bolted onto the groove of the “vertical” part of the “h” of part 102. This groove allows the “toes” to be extended or retracted to change the size of the foot as well as the springiness of the foot for differently sized amputees. The “toes” part of piece 102 is a wedge of the same polyamide plastic which allows for energy return to the amputee upon toe off in the stance cycle. The bolt that connects part 103 to part 102 allows for efficient energy transfer between the two components. The overall depth of the foot is preferably about 3″. The overall width of the foot is preferably about 1.5″. The overall length of the foot is preferably between approximately 6 and 9 inches depending on the extension of the toes.

The foot shown in FIG. 10 preferably comprises the following components: Toe piece 104; Toe piece bolts 104′ (2; not shown); Metatarsal piece 105; Bolt 1 Piece 1 106 (not shown); Bolt 1 Piece 2 107 (not shown); Squat slider Piece 1 108; Squat slider piece 2 109; Slider Set Screw 110; Squat stopper pin 111; Squat stopper pin bushing/spring assembly 112 (not shown); Rocker Plate 113; Rocker Plate Axle 114; Rocker Plate Axle Bushings 115 (2; not shown); Squat bumper 116; Squat bumper set screw 117; Squat bumper nut 118; Rubber heel strike bumper 119; Rubber heel strike set screw 120; Body 121; Male adapter 122; Male adapter screws 123 (3; not shown); and Rocker plate axle set screw 124.

The overall width of the foot is preferably about 3″ (with slider, about 1.75″ without). The overall height is about 3″. The overall length varies between approximately 7″ and 9″.

The toe piece 104 is a simple wedge of uniform width. This piece is permanently connected to the metatarsal piece 105 by the toe piece bolts 104′ and some epoxy. The metatarsal piece is preferably grooved on the bottom so the toe piece can fit in and form a flat union.

The metatarsal piece 105 is a somewhat rectangular piece with slight wedges cut off the front corners. These make the foot more rounded and less likely to catch on things. Besides the groove on the bottom for the toe piece to interface with, there are preferably a number of other features on this component. There are two ridges 130 the top side of the metatarsal piece, evenly spaced across the width of the foot. These mesh in with two grooves cut in the body 121 the foot so that the length of the foot can be adjusted. Length adjustments of the foot are controlled by those groove/ridge combinations as well as the bolt combination (parts 107 and 107) described in the next paragraph. The bolt pair is set in a slotted groove on the bottom of the metatarsal piece, which assists in the stabilization of the lengthening feature of the foot by allowing movement when loose and keeping the foot the same length when tightened. There are two grooves in the sides of the metatarsal piece which serve to decrease the weight of the foot.

Part 106 (Bolt 1 piece 1) operates as a nut. The nut looks like a screw but the threads are internal on the shaft of the screw. The head of the nut can be tightened using a flat head screwdriver. It looks like a slotted fillister captive screw without the external threads. Part 107 operates as a bolt, and is preferably a standard hex head bolt. Size of these two parts does not matter, but these preferably are approximately ¼″ in diameter (OD).

One of the features of this foot is the ability to flip a switch and squat. There are five components that create this capability in the foot. Mounted between the metatarsal piece and the body piece (in a groove cut in the body piece) are two slider plates. The first slider plate 108 is there to prevent excessive wear on the metatarsal piece and provide a solid backing for locking the squat capability in either squat mode or standing mode. This locking is provided by a set screw 110. The ability to switch between the squatting mode and the standing mode is provided by the second slider plate 109 which has a hole drilled in it appropriately placed so that when the slider is shifted fully to the “off” position, the hole is not underneath the squatter stopper pin 111, and when the slider is shifted fully to the “on” position, the hole is underneath the stopper pin, and the person can squat. The stopper pin works by either hindering or allowing the rocking of the rocker plate 113. When it is in the “off position, the rocker plate is not allowed to rock forward because the pin is prevented from dipping by the slider plate. When the slider is in the “on” position, the rocker plate is allowed to rock forward because the slider plate hole no longer prevents the stopper pin from being pushed down. When the rocker plate returns to a horizontal position, the stopper pin is returned to place by the Squat stopper pin bushing/spring assembly 112. In order to reduce the noise produced by this stopper assembly, the squat bumper 116 is made of the polyamide plastic, while all the other squat components are made from steel. The squat bumper is held in place on the rocker plate by a screw and nut 117,118 as well as a groove cut into the rocker plate.

The rocker plate 113 is the feature that allows for the ankle motion to occur. It is attached to the body of the foot via an axle 114 which is allowed to rotate in two bushings 115 and held stationary (if so desired) by a set screw 116.

The final feature of this foot is the adjustable heel bumper 119. When the rocker is rocked fully back, it hits the top of a rubber tower that serves to connect the rocker to the heel for the purpose of cushioning the heel strike portion of the stance cycle. The response of the foot to heel strike can be adjusted using the bumper set screw 120 so that the angle at which the bumper strikes can be increased or decreased.

The foot connects to the pylon with a male adapter 122 which is attached using three screws.

In order to hold the prosthesis on the leg, a suspension system is necessary. This prosthesis was designed to accommodate two different suspension systems.

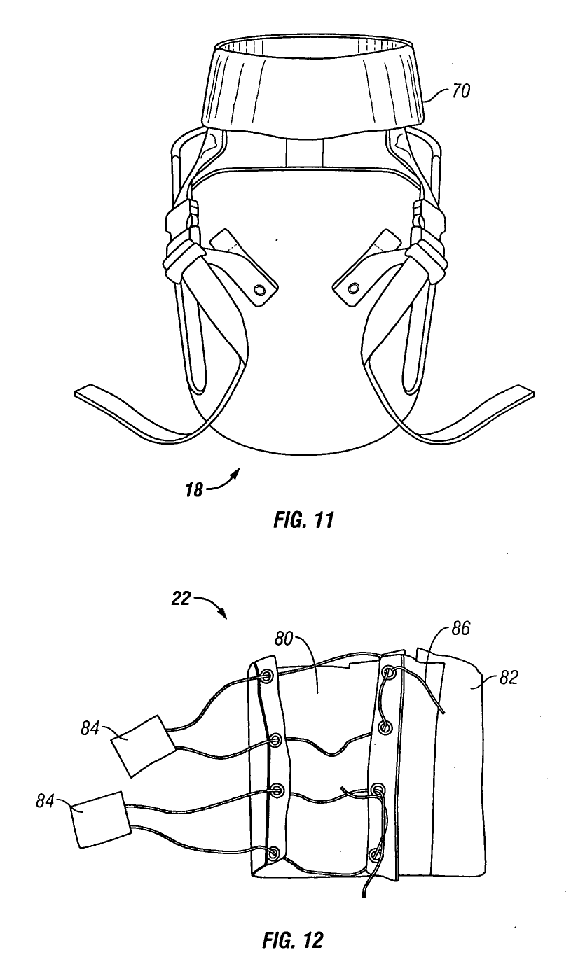

FIGS. 11-12 illustrate the preferred suspension system. The most important component is the adjustable suprapatellar strap 70 that extends from the rear of the prosthesis just behind and below the knee to the front of the knee where it attaches to a strap that binds just above the condyles of the knee. This holds the prosthesis up on the residuum. The straps are adjustable for the length of the residuum. The second component to this system is a cuff 22 that wraps around the three wings of the shell and holds them against the leg using compression. This cuff preferably has a lycra insert 80 which allows it to stretch around the prosthesis when it is first put on the residuum. The strapping system shown in FIG. 12 is then used to pull the wings together, placing the residual limb in compression, preferably comprising leather 82 and male and female hook-and-loop attachment pads 84,86. This allows friction to hold the limb in place.

Should the cuff wear out, there is another method built into this system that will allow the amputee to continue using the limb without the cuff. Holes are preferably drilled along the edges of the wings of the socket shell (FIGS. 2 and 3) that will allow the amputee to use string to bring about the same tension that the cuff created. This string method will work equally well with the suprapatellar strap system. Should the suprapatellar strap wear out, a new one can be made using leather or cloth. This can be attached to the buckles just as well as the webbing that was initially used.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

What is claimed is:

1. A lower limb prosthesis comprising:
a foot;
a pylon;
a socket;
a connector between said socket and said pylon;
an adapter between said foot and said pylon;
and a liner in said socket, said liner being substantially rigid at room temperatures and moldable at a temperature between approximately 50 degrees C. and approximately 100 degrees C.

2. The prosthesis of claim 1 wherein said socket comprises a plurality of upward extending wings.

3. The prosthesis of claim 2 wherein one of said wings is substantially wider than all other of said wings.

4. The prosthesis of claim 1 wherein said liner is moldable at a temperature between approximately 50 degrees C. and approximately 65 degrees C.

5. The prosthesis of claim 1 wherein said liner comprises an acetate or acrylic acid copolymer.

6. The prosthesis of claim 5 wherein said liner comprises a copolymer selected from the group consisting of ethylene/acrylic acid, ethylene/methacrylic acid, and ethylene/vinyl acetate.

7. The prosthesis of claim 6 wherein said liner comprises ethylene/vinyl acetate.

8. The prosthesis of claim 7 wherein said liner comprises between 10% and 50% vinyl acetate by weight.

9. The prosthesis of claim 8 wherein said liner comprises approximately 40% vinyl acetate by weight.

10. The prosthesis of claim 1 wherein said liner has a melting temperature of approximately 150 degrees C. or higher.

11. A support for a residual lower limb, said support being sized to receive the residual lower limb and comprising a socket and a liner in said socket, said liner being substantially rigid at room temperatures and moldable at a temperature between approximately 50 degrees C. and approximately 100 degrees C.

12. The support of claim 11 wherein said socket comprises a plurality of upward extending wings.

13. The support of claim 12 wherein one of said wings is substantially wider than all other of said wings.

14. The support of claim 11 wherein said liner is moldable at a temperature between approximately 50 degrees C. and approximately 65 degrees C.

15. The support of claim 11 wherein said liner comprises an acetate or acrylic acid copolymer.

16. The support of claim 15 wherein said liner comprises a copolymer selected from the group consisting of ethylene/acrylic acid, ethylene/methacrylic acid, and ethylene/vinyl acetate.

17. The support of claim 16 wherein said liner comprises ethylene/vinyl acetate.

18. The support of claim 17 wherein said liner comprises between 10% and 50% vinyl acetate by weight.

19. The support of claim 18 wherein said liner comprises approximately 40% vinyl acetate by weight.

20. The support of claim 11 wherein said liner has a melting temperature of approximately 150 degrees C. or higher.

21. The support of claim 11 additionally comprising a pylon and a connector between said socket and said pylon, said connector being adjustable for proximal, distal, and lateral alignment.

22. The support of claim 21 additionally comprising a foot and an adapter between said foot and said pylon, said foot comprising a releasable rocker mechanism, said rocker mechanism operatively configured to allow a wearer to squat.

23. The support of claim 22 wherein said adapter is adjustable for length.

23. The prosthesis of claim 1 wherein said foot comprises a releasable rocker mechanism, said rocker mechanism operatively configured to allow a wearer to squat.

24. The prosthesis of claim 1 wherein said connector is adjustable for proximal, distal, and lateral alignment.

25. The prosthesis of claim 2 wherein said plurality of upward extending wings are cantilevered at one end of the socket.

26. The prosthesis of claim 1 wherein said adapter is adjustable for length.
